(19) United States
(12) Patent Application Publication
(10) Pub. No.: US 2022/0384021 A1
(43) Pub. Date: Dec. 1, 2022

(54) EXPLANATION CREATING METHOD, EXPLANATION CREATING DEVICE, AND EXPLANATION CREATING PROGRAM

(71) Applicant: NIPPON TELEGRAPH AND TELEPHONE CORPORATION, Tokyo (JP)

(72) Inventors: Atsuhiko MAEDA, Tokyo (JP); Kenichi FUKUDA, Tokyo (JP); Sun Yeong KIM, Tokyo (JP); Yukio KIKUYA, Tokyo (JP); Kazuaki OBANA, Tokyo (JP)

(73) Assignee: NIPPON TELEGRAPH AND TELEPHONE CORPORATION, Tokyo (JP)

(21) Appl. No.: 17/775,240

(22) PCT Filed: Nov. 8, 2019
(86) PCT No.: PCT/JP2019/043956

§ 371 Date: May 6, 2022

(51) Int. Cl. G16H 40/20; G06N 5/04; G06N 5/02

(52) U.S. Cl. CPC G16H 40/20; G06N 5/045; G06N 5/022

(57) ABSTRACT

Features serving as a basis for prediction can be explained. In an explanation creating method, with regard to a prospect of each of hospitals accepting a user, prediction results are found in advance using each of features including information relating to a reason of each of the hospitals rejecting acceptance of the user. Processing prescribed in advance is performed regarding each of features to be taken as an object, out of each of the features, and finds prediction results following modification. On the basis of a difference between prediction results using each of the features and prediction results following modification found regarding the features to be taken as an object, in a case in which there is a difference that satisfies a standard set in advance regarding each of the features to be taken as an object, creating an explanation regarding prediction results relating to the reason of rejection regarding this feature.

### TECHNICAL FIELD

The technology of the disclosure relates to an explanation creating method, an explanation creating device, and an explanation creating program.

### BACKGROUND ART

When selecting a facility at which to be accepted in accordance with a user, there are cases in which searching for a facility that can accept the user is difficult. For example, a case is conceivable in which there is a request for emergency transportation from a patient, and a hospital to transport the patient to is searched for.

The number of ambulance calls is on the rise as of recent (see NPL 1). It is known that as one problem at the time of transporting a patent to a hospital by ambulance in response to a request for emergency transportation, identifying hospitals that are capable of accepting the patient is time consuming. In particular, when a transportation request is made to a hospital but is rejected thereby, and a hospital to transport the patient to has to be selected again, the time required for transportation may become markedly long.

In the flow of emergency transportation, generally, the patient is first carried into an ambulance, and thereafter a hospital to transport the patient to is searched for. Specifically, the ambulance crew members themselves search for a destination to transport the patient to, and accordingly may contact one hospital after another, or directly head to a hospital, in some cases. At this time, there are cases in which a hospital to transport the patient to is not found due to various factors such as physicians on duty, the state of examination and treatment being performed, and so forth, resulting in the patient being endlessly sent from one place to another (see NPL 2, for example).

Also, systems for making various types of prediction are being put to use in various fields, and with regard to such prediction, there is technology relating to a system for predicting the possibility of various advertisements on a Web page being clicked (e.g., NPL 3).

### CITATION LIST

### Non Patent Literature

[NPL 1] “2017 White Paper on Fire Service”, URL: https://www.fdma.go.jp/publication/hakusho/h29/chapter2/section5/45975.html Section 5 Emergency Medical System
[NPL 2] “Byouin ‘taraimawashi’ no news ni hisomu shinkokusugiru genjitsu (A far too serious reality behind the news of hospital ‘taraimawashi’ (being sent from one hospital to another))”, URL: https://limo.media/articles/-/9406
[NPL 3] Matthew Richardson, Ewa Dominowska, and Robert Ragno. 2007. Predicting clicks: estimating the click-through rate for new ads. In Proceedings of the 16th international conference on World Wide Web (WWW '07). ACM, New York, N.Y., USA, 521-530.

### SUMMARY OF THE INVENTION

### Technical Problem

Several measures to solve such a phenomenon can be conceived. For example, conceivable measures include making a list of candidates for transportation destination hospitals, with symptoms of the user, outpatient history, and so forth, as a reference, and sending an email or the like to contact a plurality of hospitals at the same time to query whether capable of accepting or not.

However, simply making a list of hospitals necessitates contacting a plurality of hospitals regarding whether capable of accepting or not, consuming time until the transportation destination hospital is finalized. Also, in a case of contacting a plurality of hospitals at the same time to query whether capable of accepting or not, there are cases in which the ambulance and the hospitals need to be connected by network, and actions need to be actively undertaken at the hospital side, leaving unresolved issues.

Also, systems that perform predictions such as in NPL 3 are often unclear in the eyes of the user regarding grounds for judgment in prediction, and there has been a problem that results of judgment made by the prediction system are not trustworthy.

The technology of the disclosure has been made with the foregoing in view, and it is an object thereof to provide an explanation creating method, an explanation creating device, and an explanation creating program that can give explanations regarding features serving as a basis for prediction.

### Means for Solving the Problem

A first aspect of the present disclosure is an explanation creating method that causes a computer to execute processing including, with regard to a prospect of each of hospitals accepting a user, prediction results are found in advance using each of features including information relating to a reason of each of the hospitals rejecting acceptance of the user, performing processing prescribed in advance regarding each of features to be taken as an object, out of each of the features, and finds prediction results following modification, and on the basis of a difference between prediction results using each of the features and prediction results following modification found regarding the features to be taken as an object, in a case in which there is a difference that satisfies a standard set in advance regarding each of the features to be taken as an object, creating an explanation regarding prediction results relating to the reason of rejection regarding this feature.

### Effects of the Invention

According to the technology of the disclosure, explanation of features serving as a basis for prediction can be given.

### BRIEF DESCRIPTION OF DRAWINGS

FIG. 1 is a block diagram illustrating a configuration of a selection supporting device according to an embodiment.

FIG. 2 is a block diagram illustrating a hardware configuration of the selection supporting device.

FIG. 3 is a diagram showing an example of history data D1.

FIG. 4 is a diagram showing an example of prediction model generation data D2.

FIG. 5 is a diagram showing an example of prediction data D3.

FIG. 6 is a diagram showing an example of score calculation data D4.

FIG. 7 is a flowchart showing a flow of learning processing by the selection supporting device.

FIG. 8 is a flowchart showing a flow of prediction processing by the selection supporting device.

FIG. 9 is a diagram showing an example of vectors representing attribute information.

FIG. 10 is a diagram showing an example of processing of the prediction data D3.

FIG. 11 is a diagram showing an example of output data containing calculated acceptance probabilities.

FIG. 12 is a flowchart showing explanation creating processing in its entirety.

FIG. 13 is a flowchart showing an example of explanatory text creating processing for each feature.

FIG. 14 is a flowchart showing an example of calculation processing for difference in prediction results in a case of classification of features being common.

FIG. 15 is a flowchart showing an example of calculation processing for difference in prediction results in a case of classification of features being separate.

FIG. 16 is a diagram showing an example showing features that are the object of analysis out of features contained in the score calculation data D4.

FIG. 17 is a diagram illustrating an example of coefficient matrices W and V.

FIG. 18 is diagram showing a conceptual representation of output of an explanatory text list.

### DESCRIPTION OF EMBODIMENTS

An example of an embodiment of technology according to the disclosure will be described below with reference to the Figures. Note that in the Figures, components and portions that are the same or equivalent are denoted by the same reference signs. Also, the dimensions and proportions in the Figures may be exaggerated for the sake of description, and differ from proportions in reality.

The present disclosure relates to prediction of facilities of which the possibility of accepting acceptance requests from users is high, to support selection of facilities at which to be accepted in accordance with requests from users, and provides technology for creating explanations for features serving as a basis for prediction.

Note that matters that were observed as a premise with regard to the technology according to the disclosure will be described. In the conventional technology described above, searching for a hospital is performed until a hospital that is capable of accepting is found. In other words, in a case in which acceptance is rejected, the processing ends once. However, it is assumed that depending on the reason for rejecting acceptance, there will be cases in which the reason is resolved by passage of time, and acceptance becomes possible. That is to say, a point was observed in that, by taking into consideration the reason for rejection, and the amount of time elapsed since the reply of the reason for rejection is received, precision of an estimation value of whether capable of accepting or not at the current point in time, and an estimation value of whether capable of accepting or not at a predetermined time, can be raised.

The configuration of the present embodiment will be described below. Note that an example of emergency transportation of a patient to a hospital, which is a facility, will be described below as an example. In a case of emergency transportation of a patient to a hospital, an ambulance crew member or an operator at a service center or the like, who is a user, selects a hospital for transportation to, and issues a transportation request (i.e., an acceptance request) to that hospital. However, this is not limited to cases of making requests to acceptance to a medical facility such as a hospital. For example, requests for acceptance of disaster victims to a shelter or the like in a disaster, requests for acceptance of materials to a facility that is a transportation destination of materials, and so forth, can be expected.

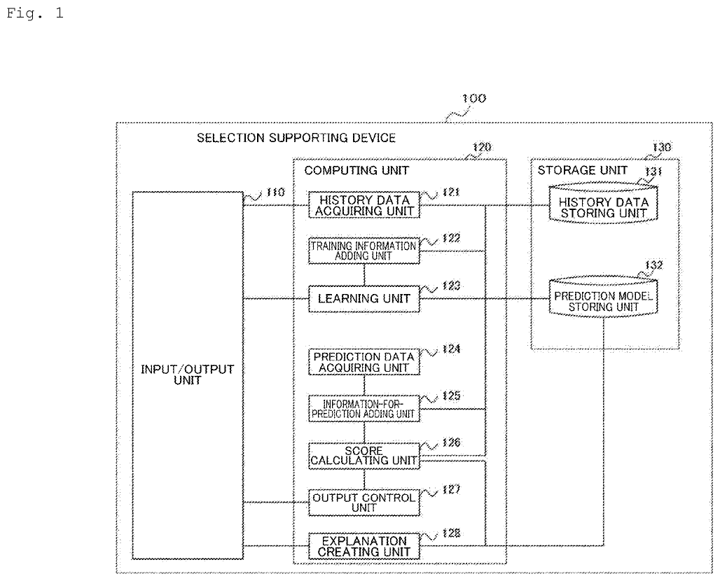

FIG. 1 is a block diagram illustrating the configuration of a selection supporting device according to the present embodiment. Note that the selection supporting device is an example of an explanation creating device according to the present disclosure.

As illustrated in FIG. 1, the selection supporting device 100 is provided with an input/output unit 110, a computing unit 120, and a storage unit 130.

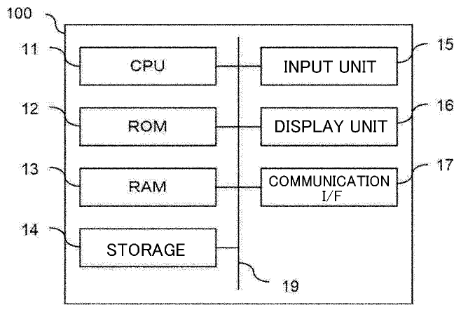

FIG. 2 is a block diagram illustrating the hardware configuration of the selection supporting device 100.

As illustrated in FIG. 2, the selection supporting device 100 has a CPU (Central Processing Unit) 11, ROM (Read Only Memory) 12, RAM (Random Access Memory) 13, storage 14, an input unit 15, display unit 16, and a communication interface (I/F) 17. The configurations are communicably connected to each other via a bus 19.

The CPU 11 is a central processing unit, and executes various types of programs, controls the parts, and so forth. That is to say, the CPU 11 reads out programs from the ROM 12 or the storage 14, and executes programs with the RAM 13 as a work region. The CPU 11 performs control of the above described configurations, and various types of computation processing, following programs stored in the ROM 12 or the storage 14. In the present embodiment, a selection support program is stored in the ROM 12 or the storage 14.

The ROM 12 stores various types of programs and various types of data. The RAM 13 temporarily stores programs and data as a work region. The storage 14 is configured of a storage device such as an HDD (Hard Disk Drive) or an SSD (Solid State Drive) or the like, and stores various types of programs including operating systems, and various types of data.

The input unit 15 includes a point device such as a mouse or the like, and a keyboard, and is used to perform various types of input.

The display unit 16 is, for example, a liquid crystal display, and displays various types of information. The display unit 16 may employ a touch panel system, and function as the input unit 15. Note that the input unit 15 and the display unit 16 correspond to the input/output unit 110.

The communication interface 17 is an interface for communicating with other equipment, such as terminal and so forth, and a standard such as, for example, Ethernet (a registered trademark), FDDI, Wi-Fi (a registered trademark), and so forth, is used.

The functional configurations of the selection supporting device 100 will be described next. The functional configurations are realized by the CPU 11 reading out the selection supporting program stored in the ROM 12 or the storage 14, loading to the RAM 13, and executing. Note that the selection supporting program is an example of an explanation creating program according to the present disclosure.

The computing unit 120 of the selection supporting device 100 includes a history data acquiring unit 121, a training information adding unit 122, a learning unit 123, a prediction data acquiring unit 124, an information-for-prediction adding unit 125, a score calculating unit 126, an output control unit 127, and an explanation creating unit 128, as illustrated in FIG. 1. The storage unit 130 includes a history data storing unit 131 and a prediction model storing unit 132.

The history data acquiring unit 121 acquires history data relating to past acceptance requests from an input device, an external database, or the, like, omitted from illustration, via the input/output unit 110, and stores in the history data storing unit 131. Acquisition of history data by the history data acquiring unit 121 is performed each time history data is accepted.

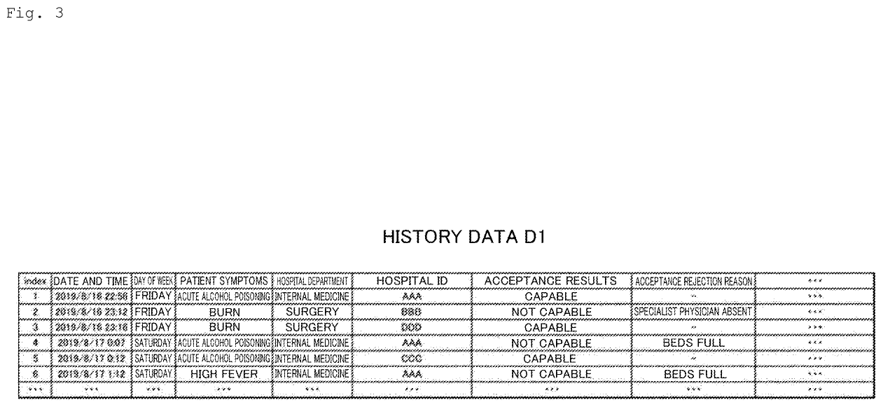

The history data storing unit 131 stores history data D1. FIG. 3 is a diagram showing an example of the history data D1. As shown in FIG. 3, the history data D1 is data in which attribute information, hospital ID, acceptance results, and acceptance rejection reason, for example, are correlated. The attribute information is various types of information relating to acceptance requests from a user. In a case of emergency transportation, the attribute information is, for example, the year, month, and date on which the request for emergency transportation was made, the day of the week thereof, the part of day thereof, symptoms of the patient, and the hospital department in accordance with the symptoms of the patient. The attribute information may also include the weather, facial complexion of the patient, heart rate, and so forth. Also, the attribute information may also include attribute information relating to candidate facilities. The hospital ID is identification information for the facility (hospital) to transport the patient to. The acceptance results are information of whether each facility accepted the acceptance request or not. The acceptance rejection reason is the rejection reason regarding when acceptance was rejected. It is sufficient for the acceptance rejection reason to be selected and input from a plurality of options prepared in advance, for example, which are the five of “in surgery”, “specialist physician absent”, “beds full”, “treatment difficult”, and “no response” here. Thus, the history data includes information relating to past acceptance requests, and information representing whether the acceptance requests were accepted or not.

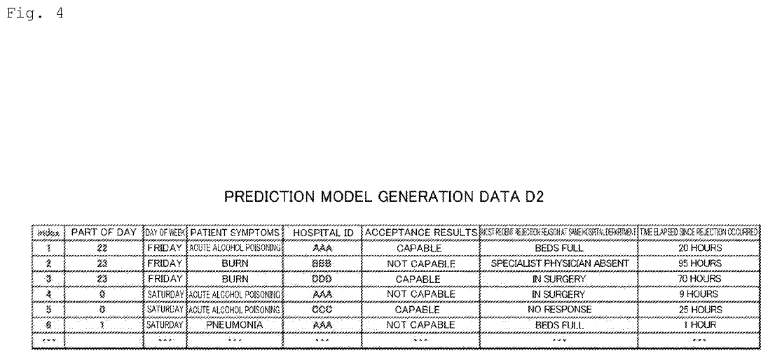

The training information adding unit 122 reads out the history data D1 stored in the history data storing unit 131, generates prediction model generation data D2 used for generating a prediction model on the basis of the history data D1, and outputs to the learning unit 123. FIG. 4 is a diagram showing an example of the prediction model generation data D2. As illustrated in FIG. 4, the prediction model generation data D2 is data relating to the part of day, the day of the week, patient symptoms, hospital ID, acceptance results, most recent rejection reason in same hospital department, and time elapsed since rejection occurred. Generation of the prediction model generation data D2 will be described later.

The learning unit 123 executes processing of performing statistical analysis using vectors and correct labels obtained from the prediction model generation data D2, learns coefficient vectors as prediction models, and stores in the prediction model storing unit 132. The learning technique of prediction models will be described later. Note that settings may be made in advance so that the processing of the learning unit 123 is executed periodically (e.g., semimonthly, monthly, etc.).

The prediction model storing unit 132 stores a prediction model. The prediction model is used to predict, on the basis of attribute information relating to a newly-occurring acceptance request, the possibility of the candidate facilities accepting the acceptance request.

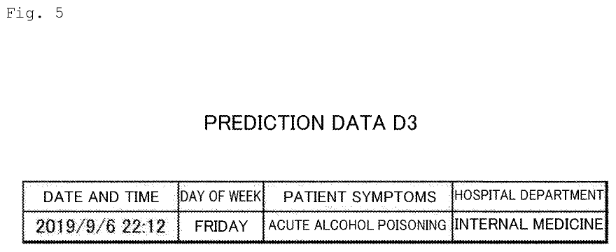

The prediction data acquiring unit 124 acquires prediction data D3 via the input/output unit 110, and outputs to the information-for-prediction adding unit 125. FIG. 5 is a diagram illustrating an example of the prediction data D3. As shown in FIG. 5, the prediction data D3 is data including, for example, the date and time of acceptance request, the day of the week, patient symptoms, and hospital department. This example shows that on Sep. 6, 2019, Friday, at 22:12, there was a patient exhibiting symptoms of acute alcohol poisoning, which is handled by the hospital department of internal medicine. In this way, attribute information relating to acceptance requests including the date and time is acquired as the prediction data D3.

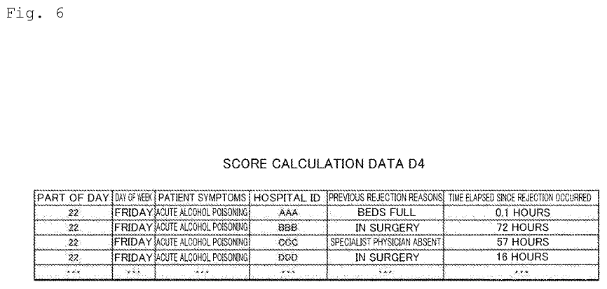

The information-for-prediction adding unit 125 generates score calculation data D4 on the basis of the acquired prediction data D3 and the history data D1 stored in the history data storing unit 131, and outputs to the score calculating unit 126. FIG. 6 is a diagram showing an example of the score calculation data D4. As shown in FIG. 6, the score calculation data D4 is data including, for example, the part of day, the day of the week, patient symptoms, hospital ID, previous rejection reasons, and time elapsed since rejection occurred. Generation of the score calculation data D4 will be described later.

The score calculating unit 126 calculates a score value for each hospital, on the basis of the score calculation data D4 output from the information-for-prediction adding unit 125 and the prediction model stored in the prediction model storing unit 132. The score value is a value representing the possibility of being accepted when an acceptance request is made to a particular facility. The calculation technique of the score value will be described later. Note that the score calculating unit 126 is an example of an estimating unit according to the present disclosure.

The output control unit 127 performs processing of creating output data on the basis of the score value calculated at the score calculating unit 126, and outputting via the input/output unit 110. The output processing will be described later.

The explanation creating unit 128 calculates, for each of the object features, the difference between prediction results according to the above-described prediction model using each of the features, and prediction results following modification that are obtained regarding the object features. On the basis of the calculated difference, the explanation creating unit 128 creates an explanation of the prediction results regarding the features, in a case in which there is a difference that satisfies a standard set in advance. The explanation of prediction results here is explanation regarding the reason why each hospital rejected acceptance of the user. Details of the explanation creating processing will be described later.

Next, operations of the selection supporting device 100 will be described. The operations of the selection supporting device 100 are an example of the estimation method according to the present disclosure.

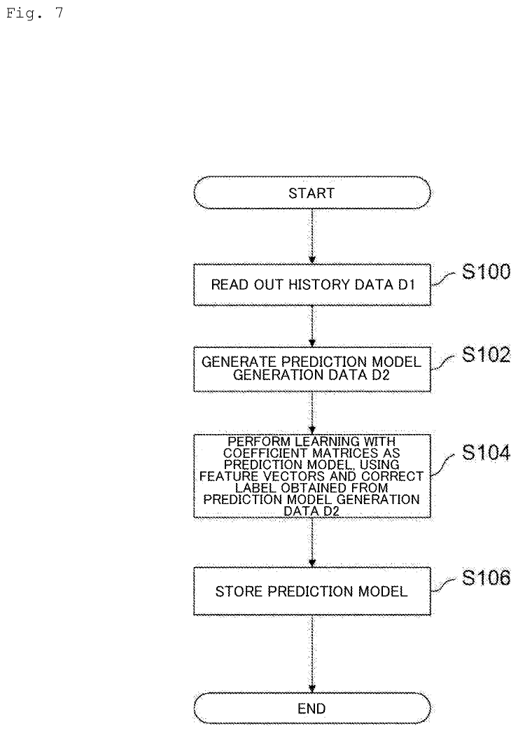

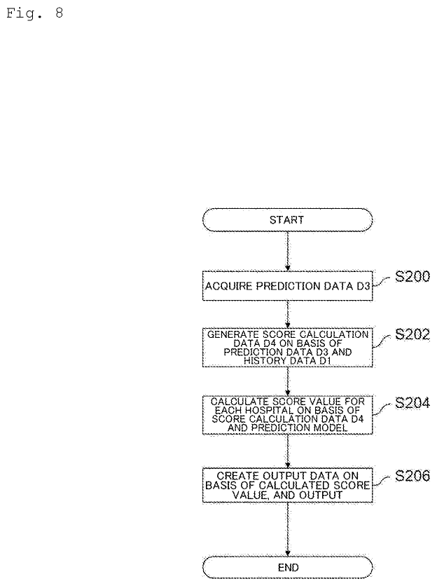

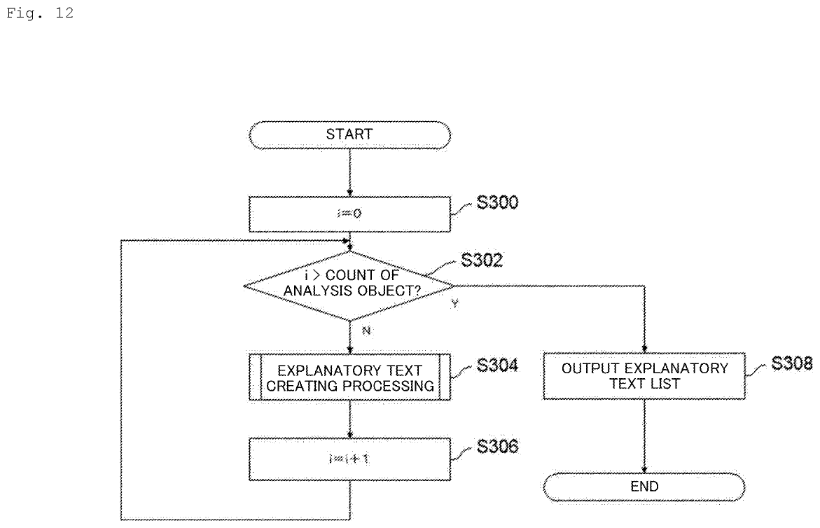

The selection supporting processing that is the operations of the selection supporting device 100 is divided into learning processing, prediction processing, and explanation creating processing, and accordingly each will be described below. FIG. 7 is a flowchart showing the flow of learning processing by the selection supporting device 100. FIG. 8 is a flowchart showing the flow of prediction processing by the selection supporting device 100. FIGS. 12 through 15 are flowcharts showing the flow of explanation creating processing. Each processing is performed by the CPU 11 reading the selection supporting program out from the ROM 12 or the storage 14, and loading to the RAM 13 and executing.

First, the learning processing of the selection supporting device 100 will be described. In the present embodiment, the selection supporting device 100 learns a prediction model by the learning processing shown in FIG. 7. The prediction model is a model for predicting the likelihood of hospitals accepting transportation requests, i.e., the possibility of accepting acceptance requests. More specifically, in the present embodiment, generating of the prediction model refers to processing for calculating a coefficient matrix. A coefficient matrix here is a matrix for calculating the score value representing the likelihood of hospitals accepting acceptance requests, and is a matrix learned as parameters with coefficients corresponding to vectors representing features. A later-described coefficient matrix W is an example of a first matrix, of which parameters that correspond to vectors representing attribute information according to the present disclosure are elements. A later-described coefficient matrix V is an example of a second matrix of which parameters that correspond to vectors representing reasons of rejection are elements. Note that learning processing may be started at an optional timing, and for example may be automatically started every certain amount of time, or may be started triggered by an operation performed by an operator. Also, the history data D1 is acquired in advance by the history data acquiring unit 121, and is stored in the history data storing unit 131.

In step S100, the CPU 11 operates as the training information adding unit 122 and reads out the history data D1 stored in the history data storing unit 131.

In step S102, the CPU 11 operates as the training information adding unit 122 generates the prediction model generation data D2 used to generate the prediction model on the basis of the history data D1, and outputs to the learning unit 123.

Generation of the prediction model generation data D2 will be described. The training information adding unit 122 performs processing of calculating elapsed time for each row in the history data D1. In order to calculate the elapsed time, first, the training information adding unit 122 references history data further in the past from the date and time of the object row, and searches rows in which acceptance rejection occurred at a date and time before the date and time of the object row for from history data of the same hospital ID and the same hospital department. For each of the rows obtained as a result of the search, the training information adding unit 122 adds, to the object row, the reason for the acceptance rejection thereof, and the calculation results of elapsed time from the date and time of the object row to the date and time of the rows obtained as the results of the search. Now, the date and time before is a most recent date and time, for example. As an example, a case of adding the acceptance rejection and elapsed time to the sixth row of D1 shown in FIG. 3 will be described. In this case, with the sixth row as the object row, the training information adding unit 122 extracts the acceptance rejection reason in the fourth row that is history data in the past from the date and time of the sixth row and that is of the same hospital ID and the same hospital department as the most recent rejection reason at the same hospital department, and calculates the elapsed time. Also, the part of day information is calculated from the date and time information in D1, and unnecessary columns are deleted as necessary. The training information adding unit 122 outputs the prediction model generation data D2 generated in this way to the learning unit 123. Note that in the learning processing, the date and time of the object row is an example of a first time according to the present disclosure, and the date and time of the row obtained as a result of the search is an example of a second time that is in the past from the first time according to the present disclosure.

In step S104, the CPU 11 operates as the learning unit 123 and learns the coefficient matrix as a prediction model, by executing processing of statistical analysis using feature vectors and correct labels obtained from the prediction model generation data D2. Note that feature vectors will be written simply as vectors hereinafter.

Learning of the prediction model will be described. In the present embodiment, the learning unit 123 takes the acceptance results column (capable/not capable) in the prediction model generation data D2 as objective variables, and executes statistical analysis with all other information as explanatory variables (vectors). The acceptance results column is the correct label representing whether the hospitals are capable of accepting or not. Thus, coefficient matrices W and V for calculating score values that represent the likelihood of being accepted by hospitals, i.e., the level of the possibility of the acceptance request being accepted, are calculated as a prediction model. For example, in a case in which the acceptance results column shows capable, labeling of 1 is performed, and in a case of not capable, labeling of 0 is performed, and this is used as objective variables to perform statistical analysis.

For the statistical analysis executed by the learning unit 123, a technique such as, for example, logistic regression analysis, ranking learning, random forest, or the like, is selected in accordance with the object. Here, a function f(p, h, Δt, r; W, V) is designed to output a great scalar value is as to the vector in a case in which the acceptance request is “acceptable”. Note that here, p, h, Δt, and r are all included in the prediction model generation data D2 as vectors or variables. W and V represent coefficient matrices of which each parameter that corresponds to a vector is an element.

An example of a case of using logistic regression will be described below. First, the following Expression (1) is an expression to find a primary score value.

[Math. 1]

sᵢⱼ=pᵢ^{T}Whⱼ+exp(−Δt_{i,j})r_{i,j}^{T}Vₕⱼ (1)

Here, pᵢ is a D×1-dimensional vector representing attribute information of the i'th patient (or patient acceptance request) occurring, and hⱼ is a vector representing, by one-hot encoding, a j count of hospitals that are acceptance candidates. Note that one-hot encoding indicates a case in which just one element represented by a vector is 1. r_{i,j} is a vector representing, by one-hot encoding, the reason for the acceptance rejection at the j'th hospital that has occurred at the most recent time, with the date and time of the acceptance request of the i'th patient occurring as a standard. Δt_{i,j} is a variable representing the elapsed time therebetween. The prediction model generation data D2 is not one-hot encoded, and accordingly encoding is performed as appropriate in cases of substituting into the above Expression (1). W and V are matrices of parameters that are to be found.

Now, the attribute information of the patient is made up of the 24 types of the part of day of issuing acceptance requests (every hour), the seven types of day of the week, and 50 types of cases, and with the number of hospitals that are acceptance candidates being 30, and reasons for acceptance rejection being five types, the details of the above Expression is as shown in Expression (2) below.

$$\begin{matrix}[ Math . 2 ] & \\ {s}_{ij} = {( \begin{matrix}{p}_{1 , \text{?}} \\ {p}_{2 , \text{?}} \\ ⋮ \\ {p}_{24 + 7 + 50 , i}\end{matrix} )}^{T} ( \begin{matrix}{w}_{1 , 1} & {w}_{1 , 2} & ⋯ & {w}_{1 , 30} \\ {w}_{2 , 1} & {w}_{2 , 2} & ⋯ & {w}_{2 , 30} \\ ⋮ & ⋮ & ⋱ & ⋮ \\ {w}_{24 + 7 + 50 , 1} & {w}_{24 + 7 + 50 , 2} & ⋯ & {w}_{24 + 7 + 50 , 30}\end{matrix} ) ( \begin{matrix}{h}_{1 , i} \\ {h}_{2 , i} \\ ⋮ \\ {h}_{30 , i}\end{matrix} ) + exp ( - {Δ l}_{i , j} ) {( \begin{matrix}{r}_{1 , i , j} \\ {r}_{2 , \text{?}} \\ ⋮ \\ {r}_{\text{?} , i , j}\end{matrix} )}^{T} ( \begin{matrix}{v}_{1 , 1} & {v}_{1 , 2} & ⋯ & {v}_{1 , 30} \\ {v}_{2 , 1} & {v}_{2 , 2} & ⋯ & {v}_{2 , 30} \\ ⋮ & ⋮ & ⋱ & ⋮ \\ {v}_{s , 1} & {v}_{s , 2} & ⋯ & {v}_{s , 30}\end{matrix} ) ( \begin{matrix}{h}_{1 , j} \\ {h}_{2 , j} \\ ⋮ \\ {h}_{30 , j}\end{matrix} ) & ( 2 )\end{matrix}$$

$$\text{?} \text{indicates text missing or illegible when filed}$$

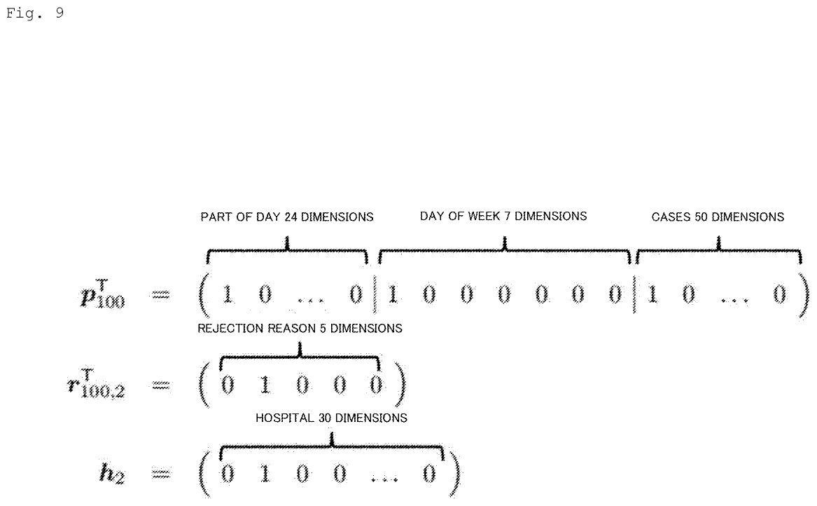

Vector pᵢ is a vector in which the part of day at which the i'th patient occurred, the day of the week of the occurrence, and patient case, are each one-hot encoded and combined. FIG. 9 is a diagram showing an example of vectors representing attribute information. The example in FIG. 9 shows an example of a vector p₁₀₀ representing attributes of a patient of index No. 100, an example of a vector r_{100,2} of the rejection reason occurring in a combination of the 100th patient and a 2nd hospital, and an example of a vector h₂ representing the 2nd hospital. Next, the coefficient matrices W and V will be described by way of example of parameters of the coefficient matrix W. With regard to a vector representing the part of day, the coefficient matrix W has parameters of w_{1,1}, w_{2,1}, . . . , w_{24,1}. In the same way, with regard to a vector representing the day of the week, the coefficient matrix W has parameters of w_{24+1,1}, w_{24+2,1}, . . . , w_{24+7,1}. The coefficient matrix W thus has parameters corresponding to vectors representing attribute information as elements thereof. In the same way, the coefficient matrix V has parameters corresponding to vectors representing the reason why hospitals rejected acceptance as elements thereof. An expression for logistic regression in which the above primary score value sᵢⱼ is substituted into the following logistic function and an acceptance probability pᵢⱼ for determining whether or not the patient will be accepted is found, is the following Expression (3).

$$\begin{matrix}[ Math . 3 ] & \\ {p}_{ij} = \frac{1}{1 + exp ( - {s}_{ij} )} & ( 3 )\end{matrix}$$

From the above expression, the probability of acceptance at each hospital regarding a patient with a particular case, occurring at a particular day of the week and a particular part of day, is found. The coefficient matrices W and V are found following the correct labels, so that the probability of acceptance is high when the correct label is capable of accepting: 1, and the probability of acceptance is low when the correct label is not capable of accepting: 0. Accordingly, the coefficient matrices W and V are learned as parameters such that take into consideration the reason for the most recent acceptance rejection, the amount of time elapsed since the most recent occurrence of the reason of rejection, or the like, for each hospital.

In step S106, the CPU 11 operates as the learning unit 123 and stores the prediction model learned as the coefficient matrices W and V in the prediction model storing unit 132.

According to the above learning processing, parameters of the first matrix W and the second matrix V are learned as the prediction model according to the present disclosure, on the basis of a function that outputs values indicating the possibility of acceptance of a user for each hospital, and correct labels. The parameters for the first matrix W and the second matrix V are learned so that in the function, a value in which the possibility of acceptance is high is output, when the user is accepted. As described above, this function includes vector (pᵢ) representing predetermined attribute information relating to acceptance requests, and the first matrix (W) of which parameters corresponding to vectors representing the attribute information are elements. This function also includes the elapsed time (Δt_{i,j}) from the second time to the first time, vectors (r_{i,j}) representing reasons why hospitals rejected acceptance, and the second matrix (V) of which parameters corresponding to vectors representing reasons of rejection as elements.

This so far has been description of operations of the learning processing of the selection supporting device 100.

Next, operations of the prediction processing of the selection supporting device 100 will be described. In the present embodiment, the selection supporting device 100 performs prediction by the prediction processing shown in FIG. 8, and supports selection made by the user. This processing is started in accordance with input of a start request (acceptance request) from an ambulance crew member or an operator at a service center for example, when there is an occurrence in which a new patient needs emergency transportation, for example.

In step S200, the CPU 11 operates as the prediction data acquiring unit 124 and acquires the prediction data D3 via the input/output unit 110, and outputs to the information-for-prediction adding unit 125.

The prediction data D3 includes attribute information relating to emergency transportation newly requested from a patient, as an acceptance request that has newly occurred. More specifically, attribute information includes, in addition to environmental information such as the date and time, the day of the week, and so forth, hospital department in accordance with symptoms of the planned transportee (patient), and patient information such as symptoms. An example of the prediction data D3 is as illustrated in FIG. 5.

In step S202, the CPU 11 operates as the information-for-prediction adding unit 125 and generates the score calculation data D4 on the basis of the acquired prediction data D3 and the history data D1 stored in the history data storing unit 131, and outputs to the score calculating unit 126.

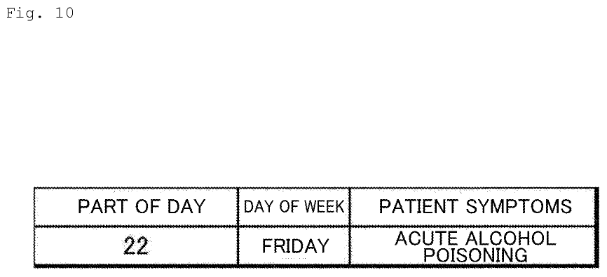

The process of generating the score calculation data D4 will be described. First, the information-for-prediction adding unit 125 converts the date and time in the prediction data D3 into part of day information as in FIG. 10, and processes unnecessary information. Next, the history data D1 stored in the history data storing unit 131 is referenced, and with regard to each hospital, a row is searched for in which the hospital department is the same as that of the prediction data D3, and also in which an acceptance rejection occurred at a date and time before than the current time (the date and time in the prediction data D3). The information-for-prediction adding unit 125 extracts the reason for the acceptance rejection from the row found as a result of the search, and calculates the elapsed time from the date and time of the row obtained as this search result to the current date and time. The date and time before than the current time is the most recent date and time, for example. As for a method of selecting a date and time other than the most recent, if a further acceptance rejection has occurred immediately prior thereto, and acceptance rejections are continuously occurring immediately therebefore for the same reason, the row with oldest time out of the continuous occurrences may be taken as the object. The reason thereof is to aim to raise prediction precision by standardizing data so as to reference as early a time as possible at which this situation in which acceptance is difficult occurred. Duplicates of the data in FIG. 10 are then created for each of the hospitals, and the data of the hospitals is combined, thereby creating the score calculation data D4, and the score calculation data D4 is output to the score calculating unit 126. An example of the score calculation data D4 is as shown in FIG. 6. Thus, the score calculation data D4 includes attribute information for each hospital, elapsed time, and reason for acceptance rejection by the hospital. Note that in the prediction processing, the date and time in the prediction data D3 is an example of the first time according to the present disclosure, and the date and time of the row obtained from the history data D1 as the search result is an example of the second time according to the present disclosure. Also, as described above, the second time is a time at which acceptance by a hospital has been rejected most recently, with the first time as a reference. Also, in a case of a plurality of times of acceptance rejection occurring within a predetermined amount of time with the second time as a standard, the time at which acceptance rejection occurred the farthest in the past within the predetermined amount of time may be set as a new second time.

In step S204, the CPU 11 operates as the score calculating unit 126, and calculates a score value for each hospital, on the basis of the score calculation data D4 output from the information-for-prediction adding unit 125 and the prediction model stored in the prediction model storing unit 132. The score value for each hospital is an example of a value indicating the possibility of acceptance for each hospital according to the present disclosure.

An example of calculating a score value will be described. First, the score calculating unit 126 acquires the coefficient matrices W and V as the prediction model stored in the prediction model storing unit 132. The attribute information, the hospital ID, and the acceptance rejection reason, in each row in the score calculation data D4, is vectorized here in accordance with the format in FIG. 9. The score value for each hospital is obtained by calculating the acceptance probability pᵢⱼ for each hospital, by applying this vector and elapsed time to the above Expression (2) and Expression (3) using the coefficient matrices W and V as the prediction model, for each hospital j. The acceptance probability represents the likelihood of requests being accepted with regard to each hospital, and the higher the acceptance probability is, this means that the more likely the acceptance request is to be accepted. As described above, a score value is found for each hospital j regarding the patient acceptance request i.

In step S206, the CPU 11 operates as the output control unit 127 and performs processing of creating output data on the basis of the calculate score values, and outputting via the input/output unit 110.

Output data will be described. For example, the output control unit 127 can create a priority list, in which a plurality of hospitals that are candidates for transporting have been prioritized by being sorted in descending order of the calculated acceptance probabilities, as output data. The calculated acceptance probabilities may be used as output data without change here, or may be used as output data in which those other than the highest ranking in the sorting are deleted. Moreover, in a case in which the distance from the location where the patient occurred to each hospital is known in advance, the hospitals to be displayed may be narrowed down by providing a threshold value for the distance, or alternatively, the distance and the acceptance probability may be displayed as a set.

According to the above prediction processing, the estimation method according to the present disclosure is executed as an estimation method for estimating the prospect, at the first time, of a hospital accepting a user. Thus, the prospect at the first time of the user being accepted is estimated by correlating the reason of the hospital rejecting acceptance of the user at the second time, and the time from the second time to the first time. Note that acceptance of the user is acceptance of some sort of action taken by the user with regard to a facility, and indicates accepting of an acceptance request issued from the user to a hospital, or the like, for example.

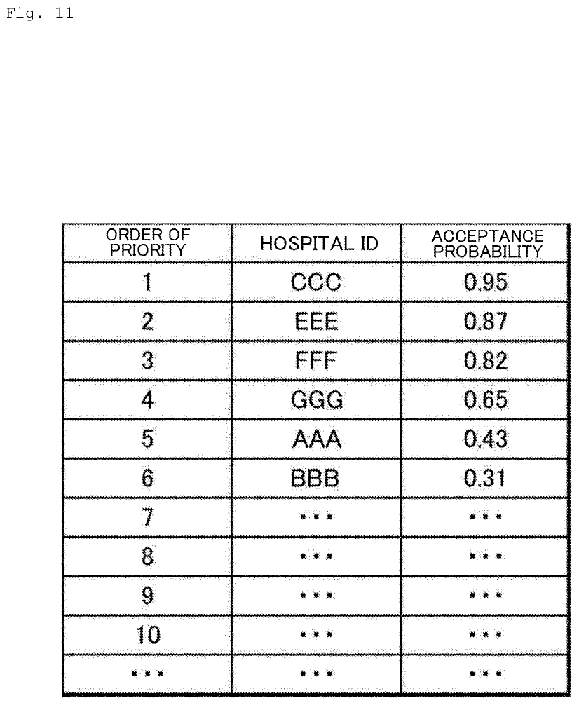

FIG. 11 is a diagram illustrating an example of output data including the calculated acceptance probability. FIG. 11 shows a priority list, sorted in descending order from hospitals with a high probability to hospitals with a low probability, on the basis of the calculated acceptance probability as output data. This indicates that the higher the acceptance probability is for the hospitals, the higher the possibility is that a transportation request will be accepted is. Accordingly, in the priority list in FIG. 11, hospital CCC that has the highest score value of 0.95 has the greatest possibility of accepting a transportation request. Also, hospital EEE that has the second highest possibility of accepting a transportation request has a score value of 0.87, and the third is hospital FFF exhibiting a score value of 0.82. By outputting this priority list as output data, an ambulance crew member or an operator, who is a user, can immediately judge that the hospital CCC has the highest possibility of accepting the current transportation request, by viewing the priority list, and issue to transportation request to the hospital CCC. Even in a case in which acceptance by the hospital CCC is rejected, the user can promptly select the second hospital EEE that is the next candidate, and accordingly the amount of time necessary of selecting candidates for transportation to can be minimized. Also, facility names may be output instead of hospital IDs, to improve user convenience.

Also, in a case of issuing a transportation request to one of the hospitals in the above, and the results are found, the ambulance crew member or operator, who is a user, immediately transmits the results to the selection supporting device 100. The history data in the history data storing unit 131 is immediately updated by the history data acquiring unit 121 in light of the results. Thus, the history data of the history data storing unit 131 is constantly maintained in a newest state, and prediction precision of a location at which to be accepted can be raised by using the newest information of acceptance rejection at each hospital.

Next, the explanation creating processing of the selection supporting device 100 will be described. In the present embodiment, an explanation of features that served as a basis for prediction is created by the explanation creating processing shown in FIGS. 12 through 15, by the selection supporting device 100, thereby supporting user selection. The features here are vectors or variables used in prediction processing.

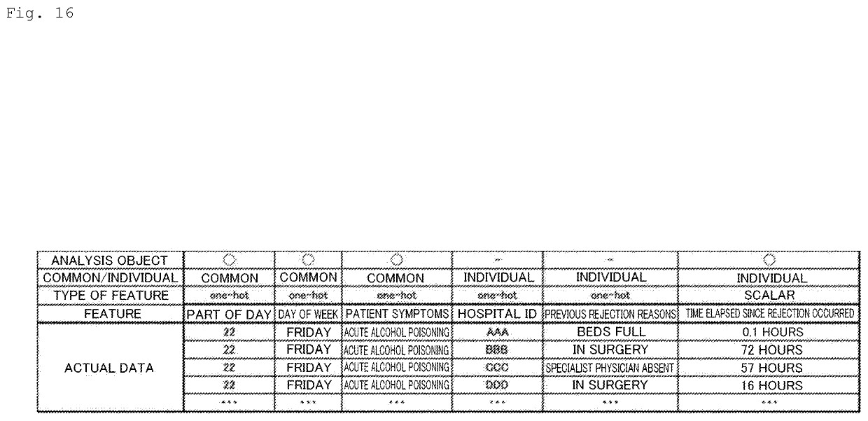

Explanation creating processing for each of the features contained in the score calculation data D4 can be performed in the explanation creating processing. FIG. 16 is a diagram showing an example showing features that are the object of analysis out of features contained in the score calculation data D4. In FIG. 16, items that are indicated by a circle under the analysis object are handled as features that are objects in the present embodiment. Regarding analysis objects, which features to take as the object for explanation creating in advance may be specified in advance. In the present embodiment, the part of day, the day of the week, patient symptoms, and time elapsed from the rejection occurring, are specified as object features. Also, common and individual classification, and type of features, are set as metadata for each of the features. With regard to common and individual classification, the part of day, the day of the week, and patient symptoms are features that are common for all rows of the score calculation data D4, i.e., for all hospitals that are prediction objects, and the time elapsed from the rejection occurring is a feature that is individual for each of the hospitals that are prediction objects. With regard to the type of the features, the part of day, the day of the week, and patient symptoms are one-hot encoded features, and the time elapsed from the rejection occurring is a scalar value. Accordingly, in a case in which the classification of features is common, the features are explainable in common to each of the hospitals, and in a case of individual, the features are explainable in accordance with each hospital.

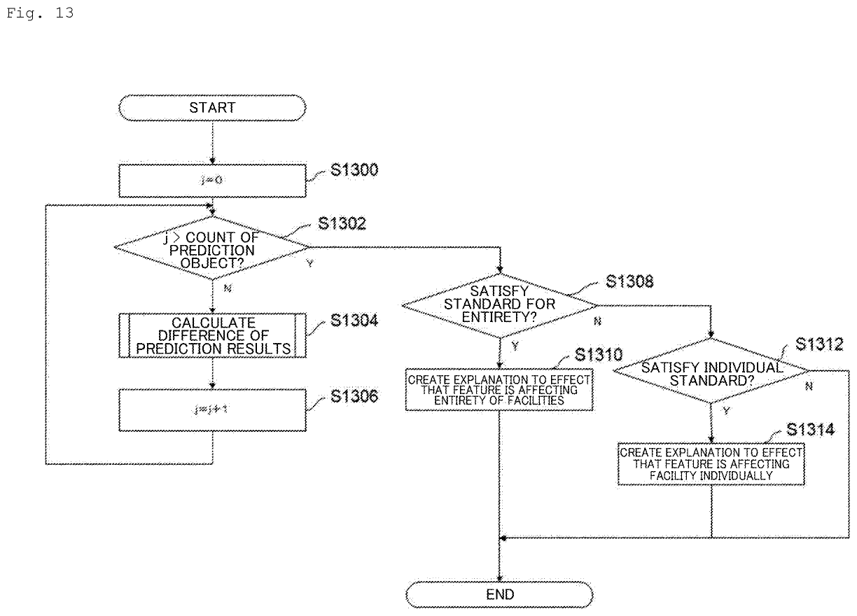

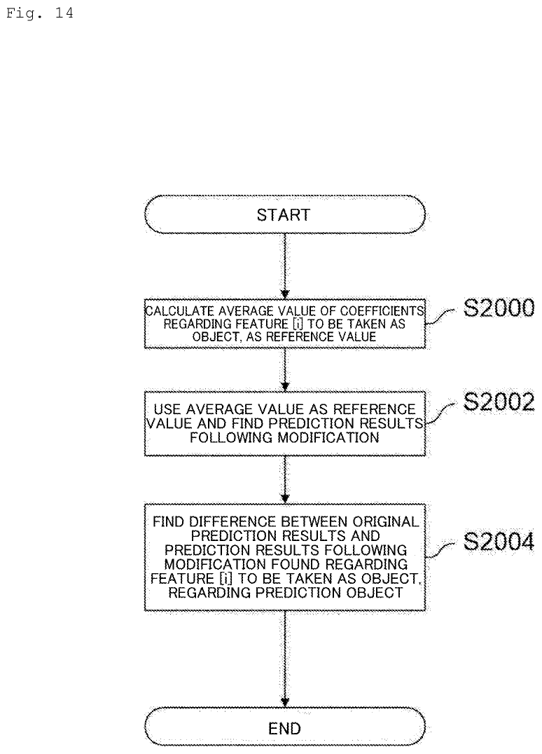

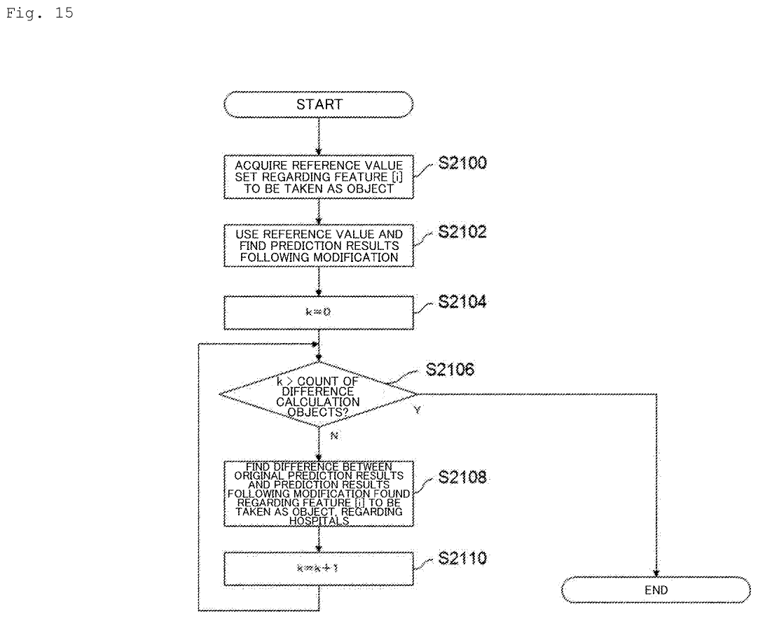

The explanation creating processing shown in FIGS. 12 through 15 will be described below. In the following, the CPU 11 functions as the explanation creating unit 128 to execute the explanation creating processing. Details of processing in step S302 in FIG. 12 are shown in FIG. 13. FIG. 14 shows processing in a case of common classification of features in detailed processing in step S1304 in FIG. 13. FIG. 15 shows processing in a case of individual classification of features in detailed processing in step S1304 in FIG. 13.

First, FIG. 12 will be described. FIG. 12 is a flowchart showing explanation creating processing in its entirety.

In step S300, the CPU 11 initializes a variable i indicating the No. of a feature to be taken as an object to 0.

In step S302, the CPU 11 determines whether or not i is greater than a count of features to be taken as objects, and in a case of being greater, transitions to step S308, and in a case of not being greater, transitions to step S304.

In step S304, the CPU 11 performs explanatory text creating processing regarding the feature [i] that is an object. In this processing, explanatory text is added to an explanatory text list only in a case in which judgment is made that the effects of individual features is great and explanatory text is necessary, on the basis of a standard set in advance regarding the features. Regarding the standard, deterioration or improvement in the probability of prediction results is set as a standard, and an explanation is created for prediction results indicating deterioration or improvement in the probability of prediction results in accordance with the standard. Accordingly, an explanation of the prediction results regarding the reason why each hospital has rejected acceptance of the user is created.

In step S306, the CPU 11 increments the variable i to i=i+1, returns to step S302, and repeats the processing.

In step S308, the CPU 11 outputs the explanatory text list via the input/output unit 110. In the present embodiment, looping is executed for the four features of the part of day, the day of the week, patient symptoms, and time elapsed from the rejection occurring, and processing for creating explanatory text is performed for each of the features.

FIG. 13 is a flowchart illustrating an example of explanatory text creating processing for each feature. Here, judgment is made from the above-described metadata regarding whether the classification of an object feature is a common feature or an individual feature, a count of prediction objects is decided, and looping processing is performed in accordance with the count of prediction objects. The prediction object is set in accordance with the classification of the feature. If a common feature, the prediction object is “each hospital”, and the count of prediction objects is set to 30, which is the count of hospitals. If an individual feature, the prediction object is the “object feature itself”, and the count of the prediction object is set to one for this feature. Specifically, in a case in which the feature [i] to be the object as described in FIG. 12 above is the features of the part of day, the day of the week, and patient symptoms, these are common for all prediction objects, and accordingly thirty loops are generated, one for each hospital. Prediction results following modification are found for each, and the difference as to the original prediction results for the relevant hospital is calculated. Also, in a case in which the feature [i] that is the object is the time elapsed from the rejection occurring, the feature is unique to each prediction object, and accordingly the prediction results following modification are found by processing one time using a reference value for the relevant hospital, rather than looping, difference as to the original prediction results for the relevant hospital is calculated. Note that as described above, the original prediction results that are the object of comparison for the difference are prediction results by prediction processing using the score calculation data D4 and the prediction model, i.e., prediction results using each of the features. The features each include information relating to the reason why each of the hospitals rejected acceptance of the user, as described above. Prediction according to the original prediction results represent the prospect of the hospital accepting the user.

In step S1300, the CPU 11 initializes a variable j that indicates the No. of the prediction object to 0.

In step S1302, the CPU 11 determines whether or not j is greater than a count of prediction objects, and in a case of being greater, transitions to step S1308, and in a case of not being greater, transitions to step S1304. In the present embodiment, in a case in which the classification of the feature [i] to be taken as an object is a common feature, the count of prediction objects is 30, which is the count of hospitals, and in a case in which the classification of the feature [i] to be taken as an object is an individual feature, the count of prediction objects is one.

In step S1304, the CPU 11 finds the prediction results following modification for the prediction object, and calculates the difference between the original prediction results and the prediction results following modification. Details of the processing of this step will be described later in FIGS. 14 and 15, since the processing differs depending on whether a case in which the classification of the features is common or a case in which the classification of the features is individual. An example of one-hot encoding regarding case of common classification, and an example of a scalar value regarding case of individual classification, will be described respectively.

In step S1306, the CPU 11 increments the variable j to j=j+1, returns to step S1302, and repeats the processing.

In step S1308, in a case in which the feature [i] to be taken as the object satisfies the standard for the entirety of hospitals, the CPU 11 transitions to step S1310, and in a case of not satisfying the standard, transitions to step S1312.

In step S1310, the CPU 11 creates an explanation to the effect that the feature is affecting the entirety of hospitals. The explanation created in this step S1310 and S1314 will be described later.

In step S1312, in a case in which the feature [i] to be taken as the object satisfies an individual standard for a hospital, the CPU 11 transitions to step S1314, and in a case of not satisfying the standard, this processing ends (transitions to step S306).

In step S1314, the CPU 11 creates an explanation to the effect that the feature is affecting a hospital individually.

In this way, the processing of step S1304 is performed for each of the features to be taken as an object, as a set with prediction objects corresponding to classifications including common and individual. Note that while determination is not made regarding individual standards in a case of satisfying an entirety standard, this is not limiting. For example, in a case of satisfying an entirety standard, following step S1310, determination of an individual standard may be performed in step S1312, and an individual explanation may be created.

FIG. 14 is a flowchart showing an example of calculation processing for difference in prediction results in a case of classification of features being common. The following processing is executed for each hospital of prediction object [j]. Note that specific processing techniques of each step will be described later.

In step S2000, the CPU 11 calculates an average value of coefficients as a reference value regarding the feature [i] to be taken as the object, for the prediction object.

In step S2002, the CPU 11 finds prediction results following modification for the prediction object, using the average value calculated with regard to the feature [i] to be taken as the object as a reference value.

In step S2004, the CPU 11 finds, with regard to the prediction object, the difference between the prediction results by the above-described prediction processing and the prediction results following modification found regarding the feature [i] to be taken as the object.

FIG. 15 is a flowchart showing an example of calculation processing for difference in prediction results in a case of classification of features being individual. Note that specific processing techniques of each step will be described later.

In step S2100, the CPU 11 acquires the reference value set regarding the features [i] to be taken as the object.

In step S2102, the CPU 11 finds prediction results following modification, using the reference value acquired regarding the features [i] to be taken as the object. The prediction results following modification here are the prediction results regarding each of the hospitals.

In step S2104, the CPU 11 initializes a variable indicating the No. of difference calculation object [k] to 0. The difference calculation object here is hospitals, and the count of the difference calculation object is the count of hospitals, which is 30.

In step S2106, the CPU 11 determines whether or not k is greater than a count of difference calculation objects, and in a case of being greater, ends this processing, and in a case of not being greater, transitions to step S2108.

In step S2108, the CPU 11 finds, with regard to the hospitals that are the difference calculation objects [k], the difference between the original prediction results by the above-described prediction processing and the prediction results following modification of the hospitals, found regarding the feature [i] to be taken as the object.

In step S2110, the CPU 11 increments the variable k to k+1, returns to step S2106, and repeats the processing.

The contents of the individual processing steps in the above explanation creating processing will be described in further detail.

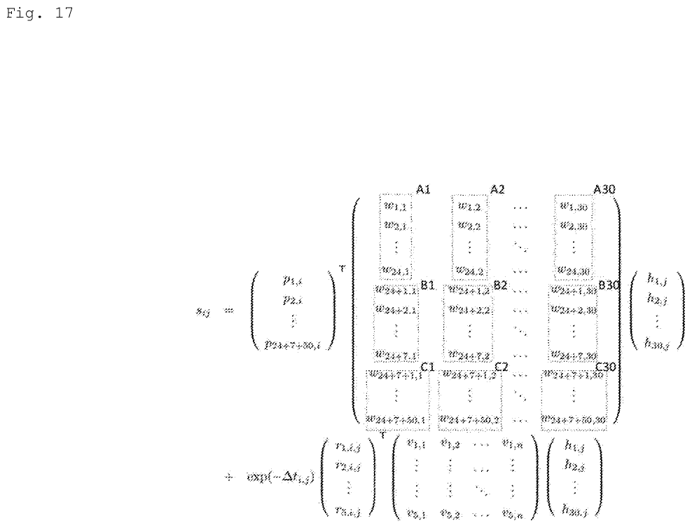

Details of processing in a case in which the classification of features is common (here, a case in which the type of features is one-hot encoded features) in the above-described steps S2000 through S2004 will be described here. FIG. 17 is a diagram illustrating an example of coefficient matrices W and V. In a case in which the hospital of index No. 1 is the prediction object, A1, B1, and C1 are relevant coefficients in the coefficient matrix W, as illustrated in FIG. 17. In the same way, for the hospital of index No. 2, A2, B2, and C2 are relevant coefficients. For the hospital of index No. 30, A30, B30, and C30 are relevant coefficients. Further, in a case in which the hospital of index No. 1 is the prediction object, and also the feature that is the object is the part of day, A1 is the relevant coefficients. In this case, the average value of the coefficients of A1 is calculated, prediction results are found with that value, and the difference between this value and the original prediction results using the score calculation data D4 is calculated.

For example, in a case in which the score calculation data is p₁₀₀ in FIG. 6, and the hospital that is the object of prediction is hospital h₂ of index No. 2, the parameters within the coefficient matrix W used to find the prediction results of the acceptance probability is the following Expression (4). Note that while there is the coefficient matrix Vas well, this will be omitted here.

[Math. 4]

(w_{1,2},w_{24+1,2},w_{24+7+1,2}) (4)

Conversely, in a case of using the average value of all coefficients corresponding to one-hot encoded data corresponding to the day of the week, and rest is the same as the conditions described above, for finding the acceptance probability, the parameters used for prediction are changed to the following Expression (5). Note that while there is the coefficient matrix V as well, this will be omitted here.

[Math. 5]

((w_{1,2}+w_{2,2}+w_{3,2}+w_{4,2}+w_{5,2}+w_{6,2}+w_{7,2})/7,w_{24+1,2}+w_{24+7+1,2}) (5)

Further, in order to further clarify the explanation of the features that served as a basis for prediction, the aforementioned average value of coefficients may be an average value of coefficients excluding only coefficients corresponding to the day of the week included in the prediction data D3. For example, in a case in which the coefficient corresponding to Friday included in D3 is w_{6,2}, the parameters used for prediction may be changed to the following Expression (6).

((w_{1,2}+w_{2,2}+w_{3,2}+w_{4,2}+w_{6,2}+w_{7,2})/6,w_{24+2,2}+w_{24+7+1,2}) (6)

Thus, prediction results following modification with regard to a total of 30 or one prediction objects are found with regard to the features to be taken as the object.

Now, the standard for the entirety in step S1308 and the individual standard in step S1312 will be described. With regard to the standard for the entirety, if the feature is common to the prediction objects, for example, the standard is that there has been decrease (or increase) in acceptance possibility by a certain amount (e.g., 10% or more) at a certain proportion or more (e.g., 24, which is 80%) of the total 30. In a case in which the calculated difference satisfies this standard, explanatory text to the effect that the overall acceptance possibility is decreasing (or increasing) due to the value of the feature that is the object is added to the explanatory text list in step S1310.

Alternatively, even if the acceptance possibility is not decreasing at 80% of the entirety, the standard is that there is a decrease in acceptance possibility by a certain amount or more (e.g., 10% or more) at individual prediction objects regarding a feature that is in common to the prediction objects, as an individual standard. In a case in which the calculated difference satisfies this standard, explanatory text to the effect that the acceptance possibility is decreasing at that hospital due to the value of that feature is added to the explanatory text list in step S1314. In a case in which the feature is not common to the prediction objects, the step of step S1308 will return N without fail.

For example, an assumption will be made that in a case in which the feature of “patient symptoms” in the score calculation data D4 is “acute alcohol poisoning”, the acceptance possibility is lower than average “patient symptoms” at 80% of the hospitals. In this case, explanatory text of “Overall acceptance possibility decreases in cases of acute alcohol poisoning.” is added to the explanatory text list.

Also, in a case in which the feature “part of day” in the score calculation data D4 is “0” for instance, and if the acceptance possibility is 10% or more lower than average parts of the day only at the hospital of index No. 2 (tentatively, hospital EEE), explanatory text of “Possibility of acceptance is lower at this part of day than normal at hospital EEE.” is added to the explanatory text list.

The above is description of processing relating to one-hot encoded features in steps S2000 through S2004. As described above, in a case in which the type of the features to be taken as an object is one-hot encoded, an average value of coefficients of the feature to be taken as an object is used as a reference value, and prediction results following modification are found.

Next, details of processing in a case in which the classification of features is individual (a case in which the type of feature is a scalar value feature here) in steps S2100 through S2108 will be described. Details of processing regarding scalar value features will be described. In a case of a scalar value, a reference value is set in advance for each feature. It is sufficient to find and set in advance a value that is an average value or a normal value for the scalar value to serve as the reference value.

For example, “time elapsed from rejection occurring” is a scalar value, and six hours is set as a reference value. In this case, even if the value is 0.1 hours in the score calculation data D4, this is modified to a value of six hours, prediction results following modification are obtained for each of the hospitals, and difference as to the original prediction results is found. Here, it is sufficient to modify the Δt_{i,j} in the above-described Expression (2) to the reference value, and find the prediction results following modification. The prediction results following modification are found for each of the hospitals, and the differences as to the original prediction results are calculated for each.

Processing to add explanatory text is performed for scalar values as well, if a certain amount or greater (e.g., 10% or more). The case of “time elapsed from rejection occurring” is not a feature common to the prediction objects, and accordingly step S1308 returns N. If there is difference in prediction results of a predetermined amount or more in the calculation object of individual differences in step S1312, processing to create explanation data is performed.

In a case in which the type of the feature that is the object is scalar value, prediction results following modification are found using the reference value set in advance for this feature to be taken as the object.

For example, in the prediction object in each row in the score calculation data D4, “time elapsed from rejection occurring” is modified to six hours, which is the reference value. The difference between the prediction results following modification and the original prediction results is found for each of the hospitals, and in a case in which decrease of 10% or more was observed only for the 0.1 hours of the first row, explanatory text of “Acceptance possibility is decreasing at hospital AAA due to beds becoming full 0.1 hours ago.” is added to the explanatory text list.

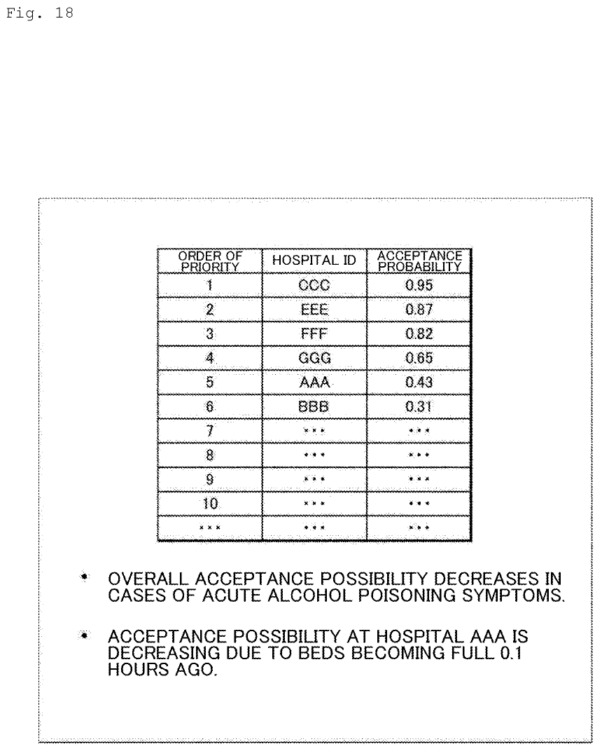

FIG. 18 is a diagram showing a conceptual representation of output of an explanatory text list. It is sufficient to add the above-described explanatory text to the output data, as illustrated in FIG. 18.

As described above, according to the selection supporting device 100 of the present embodiment, explanation of features serving as a basis for prediction is enabled.

Although an example has been described in the above embodiment regarding a case of creating explanatory text regarding prediction results of predicting the prospect of an acceptance request of a user being accepted at a facility that is a hospital, this example is not limiting. For example, application may also be made to a case of replacing the facilities with Web advertisements, setting individual features as some sort of attributes of the Web advertisement side, and finding the probability of a user having a certain attribute clicking on each Web advertisement.

Note that various types of processors other than a CPU may execute the learning processing, the prediction processing, or the explanation creating processing that the CPU executes by reading in software (programs) and executing in the above embodiments. Examples of processors in this case include dedicated electric circuits and so forth that are processors having a circuit configuration designed for dedicated execution of particular processing, such as PLDs (Programmable Logic Device) of which the circuit configuration can be changed after manufacturing, like FPGAs (Field-Programmable Gate Array) and so forth, and ASICs (Application Specific Integrated Circuit) and so forth. This learning processing, prediction processing, or explanation creating processing may also be executed by one of these various types of processors, or may be executed by a combination of two or more processors of the same type or different types (e.g., a plurality of FPGAs, a combination of a CPU and an FPGA, and so forth). More specifically, the hardware configuration of these various types of processors are electric circuits where circuit elements such as semiconductor elements and so forth are combined.

Also, while description is made in the above embodiments regarding an arrangement in which a selection supporting program is stored (installed) in the storage 14 in advance, this is not limiting. The program may be provided in a form stored in a non-transitory storage medium, such as a CD-ROM (Compact Disk Read Only Memory), DVD-ROM (Digital Versatile Disk Read Only Memory), USB (Universal Serial Bus) memory, and so forth. A form may also be made in which the program is downloaded from an external device via a network.

In relation to the above embodiment, the following appendices are further disclosed.

### APPENDIX 1

An explanation creating device, configured including:

a memory, and

at least one processor connected to the memory,

wherein the processor,

with regard to a prospect of each of hospitals accepting a user, prediction results are found in advance using each of features including information relating to a reason of each of the hospitals rejecting acceptance of the user,

performs processing prescribed in advance regarding each of features to be taken as an object, out of each of the features, and finds prediction results following modification, and

on the basis of a difference between prediction results using each of the features and prediction results following modification found regarding the features to be taken as an object, in a case in which there is a difference that satisfies a standard set in advance regarding each of the features to be taken as an object, creates an explanation regarding prediction results relating to the reason of rejection regarding this feature.

### APPENDIX 2

A non-transitory storage medium storing an explanation creating program that causes a computer to execute,

with regard to a prospect of each of hospitals accepting a user, prediction results are found in advance using each of features including information relating to a reason of each of the hospitals rejecting acceptance of the user,

performing processing prescribed in advance regarding each of features to be taken as an object, out of each of the features, and finds prediction results following modification, and

on the basis of a difference between prediction results using each of the features and prediction results following modification found regarding the features to be taken as an object, in a case in which there is a difference that satisfies a standard set in advance regarding each of the features to be taken as an object, creating an explanation regarding prediction results relating to the reason of rejection regarding this feature.

### REFERENCE SIGNS LIST

100 Selection supporting device
110 Input/output unit
120 Computing unit
121 History data acquiring unit
122 Training information adding unit
123 Learning unit
124 Prediction data acquiring unit
125 Information-for-prediction adding unit
126 Score calculating unit
127 Output control unit
128 Explanation creating unit
130 Storage unit
131 History data storing unit
132 Prediction model storing unit

1. An explanation creating method comprising:
determining prediction results using each of features including information relating to a reason of each of the hospitals rejecting acceptance of the user as a prospect of each of hospitals accepting a user;
performing processing prescribed in advance regarding each of features to be taken as an object, out of each of the features, and finds prediction results following modification; and
creating, based on a difference between prediction results using each of the features and prediction results following modification found regarding the features to be taken as an object, and further based on a difference that satisfies a standard set in advance regarding each of the features to be taken as an object, an explanation regarding prediction results relating to the reason of rejection regarding this feature.

2. The explanation creating method according to claim 1,
wherein the processing prescribed in advance is performed for each of the features to be taken as an object, as a set with prediction objects corresponding to classifications including common and individual,
wherein a standard is set for each of the hospitals, for the entirety or individually,
wherein, in a case of satisfying a standard for the entirety out of standards, an explanation is created such that a feature is affecting the entirety of hospitals, and
wherein, in a case of satisfying an individual standard out of the standards, an explanation is created such that a feature is individually affecting a hospital.

3. The explanation creating method according to claim 1,
wherein types of features are a case of one-hot encoded in which just one element represented by a vector is 1, and a case of a scalar value that is a value corresponding to a feature,
wherein in a case in which the type of the feature that is to be taken as the object is the one-hot encoded, the prediction results following modification are found using an average value of coefficients regarding the feature that is to be taken as the object or an average value of coefficients in which predetermined coefficients are excluded, as a reference value,
and wherein in a case in which the type of the feature that is to be taken as the object is the scalar value, the prediction results following modification are found using a reference value set in advance regarding the feature that is to be taken as the object.

4. The explanation creating method according to claim 1,
wherein, regarding the standards, decrease or increase of probability of the prediction results is set as the standards,
and wherein an explanation is created for the prediction results indicating the decrease or increase of probability in the prediction results in accordance with the standards.

5. An explanation creating method comprising:
determining prediction results using each of predetermined features;
performing processing prescribed in advance regarding each of features to be taken as an object, out of each of the features, and finds prediction results following modification; and
creating, based on a difference between prediction results using each of the predetermined features and prediction results following modification found regarding the features to be taken as an object, and further based on a difference that satisfies a standard set in advance regarding each of the features to be taken as an object, an explanation regarding prediction results regarding this feature.

6. An explanation creating device comprising a processor configured to execute a method comprising:
determining, with regard to a prospect of each of hospitals accepting a user, prediction results using each of features including information relating to a reason of each of the hospitals rejecting acceptance of the user;
performing processing prescribed in advance regarding each of features to be taken as an object, out of each of the features, and finds prediction results following modification, and
creating, based on a difference between prediction results using each of the features and prediction results following modification found regarding the features to be taken as an object, and further based on a difference that satisfies a standard set in advance regarding each of the features to be taken as an object, an explanation regarding prediction results relating to the reason of rejection regarding this feature.

7. (canceled)

8. The explanation creating method according to claim 2,
wherein types of features are a case of one-hot encoded in which just one element represented by a vector is 1, and a case of a scalar value that is a value corresponding to a feature,
wherein in a case in which a type of the feature that is to be taken as the object is the one-hot encoded, the prediction results following modification are found using an average value of coefficients regarding the feature that is to be taken as the object or an average value of coefficients in which predetermined coefficients are excluded, as a reference value,
and wherein in a case in which the type of the feature that is to be taken as the object is the scalar value, the prediction results following modification are found using a reference value set in advance regarding the feature that is to be taken as the object.

9. The explanation creating method according to claim 2,
wherein, regarding the standards, decrease or increase of probability of the prediction results is set as the standards,
and wherein an explanation is created for the prediction results indicating the decrease or increase of probability in the prediction results in accordance with the standards.

10. The explanation creating method according to claim 3,
wherein, regarding the standards, decrease or increase of probability of the prediction results is set as the standards, and
wherein an explanation is created for the prediction results indicating the decrease or increase of probability in the prediction results in accordance with the standards.

11. The explanation creating method according to claim 5,
wherein the processing prescribed in advance is performed for each of the features to be taken as an object, as a set with prediction objects corresponding to classifications including common and individual,
wherein, regarding the standards, a standard is set for each of the hospitals, for the entirety or individually,
wherein, in a case of satisfying a standard for the entirety out of the standards, an explanation is created such that a feature is affecting the entirety of hospitals, and
wherein, in a case of satisfying an individual standard out of the standards, an explanation is created such that a feature is individually affecting a hospital.

12. The explanation creating method according to claim 5,
wherein types of features are a case of one-hot encoded in which just one element represented by a vector is 1, and a case of a scalar value that is a value corresponding to a feature,
wherein in a case in which the type of the feature that is to be taken as the object is the one-hot encoded, the prediction results following modification are found using an average value of coefficients regarding the feature that is to be taken as the object or an average value of coefficients in which predetermined coefficients are excluded, as a reference value, and
wherein in a case in which the type of the feature that is to be taken as the object is the scalar value, the prediction results following modification are found using a reference value set in advance regarding the feature that is to be taken as the object.

13. The explanation creating method according to claim 5,
wherein, regarding the standards, decrease or increase of probability of the prediction results is set as the standards, and
wherein an explanation is created for the prediction results indicating the decrease or increase of probability in the prediction results in accordance with the standards.

14. The explanation creating method according to claim 11,
wherein types of features are a case of one-hot encoded in which just one element represented by a vector is 1, and a case of a scalar value that is a value corresponding to a feature,
wherein in a case in which the type of the feature that is to be taken as the object is the one-hot encoded, the prediction results following modification are found using an average value of coefficients regarding the feature that is to be taken as the object or an average value of coefficients in which predetermined coefficients are excluded, as a reference value, and
wherein in a case in which the type of the feature that is to be taken as the object is the scalar value, the prediction results following modification are found using a reference value set in advance regarding the feature that is to be taken as the object.

15. The explanation creating method according to claim 11,
wherein, regarding the standards, decrease or increase of probability of the prediction results is set as the standards, and
wherein an explanation is created for the prediction results indicating the decrease or increase of probability in the prediction results in accordance with the standards.

16. The explanation creating method according to claim 14,
wherein, regarding the standards, decrease or increase of probability of the prediction results is set as the standards,
and wherein an explanation is created for the prediction results indicating the decrease or increase of probability in the prediction results in accordance with the standards.

17. The explanation creating device according to claim 6,
wherein the processing prescribed in advance is performed for each of the features to be taken as an object, as a set with prediction objects corresponding to classifications including common and individual,
wherein, regarding the standards, a standard is set for each of the hospitals, for the entirety or individually,
wherein, in a case of satisfying a standard for the entirety out of the standards, an explanation is created such that a feature is affecting the entirety of hospitals,
and wherein, in a case of satisfying an individual standard out of the standards, an explanation is created such that a feature is individually affecting a hospital.

18. The explanation creating device according to claim 6,
wherein types of features are a case of one-hot encoded in which just one element represented by a vector is 1, and a case of a scalar value that is a value corresponding to a feature,
wherein in a case in which the type of the feature that is to be taken as the object is the one-hot encoded, the prediction results following modification are found using an average value of coefficients regarding the feature that is to be taken as the object or an average value of coefficients in which predetermined coefficients are excluded, as a reference value,
and wherein in a case in which the type of the feature that is to be taken as the object is the scalar value, the prediction results following modification are found using a reference value set in advance regarding the feature that is to be taken as the object.

19. The explanation creating device according to claim 6,
wherein, regarding the standards, decrease or increase of probability of the prediction results is set as the standards,
and wherein an explanation is created for the prediction results indicating the decrease or increase of probability in the prediction results in accordance with the standards.

20. The explanation creating device according to claim 17,
wherein types of features are a case of one-hot encoded in which just one element represented by a vector is 1, and a case of a scalar value that is a value corresponding to a feature,
wherein in a case in which the type of the feature that is to be taken as the object is the one-hot encoded, the prediction results following modification are found using an average value of coefficients regarding the feature that is to be taken as the object or an average value of coefficients in which predetermined coefficients are excluded, as a reference value,
and wherein in a case in which the type of the feature that is to be taken as the object is the scalar value, the prediction results following modification are found using a reference value set in advance regarding the feature that is to be taken as the object.
